(19) United States
(12) Patent Application Publication
(10) Pub. No.: US 2022/0331544 A1
(43) Pub. Date: Oct. 20, 2022

(54) INTELLIGENT AUTOMATIC OXYGEN THERAPY SYSTEM

(71) Applicant: Volker Karel Espinoza Torres, Quito (EC)

(72) Inventor: Volker Karel Espinoza Torres, Quito (EC)

(21) Appl. No.: 17/578,887

(22) Filed: Jan. 19, 2022

(30) Foreign Application Priority Data
Nov. 20, 2020 (EC) SENADI-2020-75039

(51) Int. Cl. A61M 16/10; A61M 16/00; A61B 5/1455

(52) U.S. Cl. CPC A61M 16/1005; A61M 16/024; A61B 5/14551; A61M 2230/205; A61M 2202/0208; A61M 2016/1025

(57) ABSTRACT

The Intelligent Automatic Oxygen Therapy System provides a device that allows the automatic and intelligent dosage of the percentage of an oxygen/air gas mixture and the flow delivered to each patient through non-invasive oxygen therapy procedures, based on the analysis of several measured variables that confirm the SpO2 value before taking any action. This device allows to measure biomedical signs with the main object of monitoring the oxygen saturation (SpO2), confirming its value with the analysis of the mentioned signs according to their concordance and interrelationship with each other. The equipment through artificial intelligence detects events that can occur due to the movement or for misplaced sensors. It also keeps and analyzes the records of the patient, evaluates the alarms in an intelligent way by correlating all acquired data; with this analysis the equipment can automatically and reliably provide the oxygen/air mixture adequate for each patient, with five operation modes. It activates the processed and valid alarms in an intelligent way and informs in a timely manner to the personal staff about possible found pathologies.

### FIELD OF THE INVENTION

The present invention refers to devices, apparatus, equipment, or medical systems for measurement and automatic control of oxygenation. The device in some embodiments allows measuring: oxygen saturation, heart rate, form the plethysmographic wave, form the wave, and to obtain the respiratory rate value, form the wave and values of the ECG, capnography, non-invasive blood pressure, and temperature. Based on this information, values are analyzed, and the oxygen therapy gas flow is automatically controlled. Besides, it keeps and analyzes changes in the answer of each patient so that to auto-adjust the regulation through an intelligent system. In some embodiments it analyzes and controls intelligently the alarms of the system for its optimization and activation.

### BACKGROUND OF THE INVENTION

Provision of oxygen flow in oxygen therapy needs a constant regulation which currently is performed manually through the supervision of alarms set in general in determined ranges and not necessarily for the particular condition of the patient. Being this a time-consuming activity, many times they are unattended due to the number of patients under supervision of the same nursing staff, being this added to the number of false alarms which reduce the reliability of the systems.

Unfortunately, an inappropriate provision of oxygen therapy and especially the amount of oxygen administered both in excess and in quantities lower than those necessary for the organism may cause irreversible damages in the organism. One of the most evident cases is retinopathy of prematurity (ROP) which causes visual loss due to the excessive provision of oxygen. On the other hand, low levels of oxygen may cause irreversible damages to certain organs. An exposition to low level of oxygen in less than 1 minute may damage the cerebral cortex, in 10 minutes the liver and kidney, and in 2 hours the skeletal muscles.

U.S. Pat. No. 7,222,624 provides methods and apparatus to administer respiratory oxygen to a patient, wherein the oxygen flow is controlled to control and maintain the response to the patient's pulse rate and the hemoglobin saturation in the blood.

Document ES2743025 provides a monitoring device of an oxygen therapy which comprises: a gas flow path module having one input connector and one output connector which defines the trajectory of the gas flow adapted to pass a gas flow from a source to a breathing interface for a person, the trajectory of the gas flow has various successive sections from the input connector to the output connector, these sections including: a flow conditioner; a nozzle, and an oscillation chamber adapted to induce an oscillation in the gas flow that varies as a function of a flow rate of the gas flow, the oscillation chamber having a volume smaller than 1000 mm³; and an measurement arrangement adapted to measure the oscillation induced in the gas flow and to determine the flow rate on the basis of the oscillation that is measured.

U.S. Pat. No. 5,957,885 describes a patient care system comprising an interface unit, a Patient Controlled Analgesia (PCA) unit, and a pulse oximetry unit. The PCA unit provides PCA administration, including administration of a dose of analgesic upon request, and the pulse oximetry unit provides constant monitoring of the patient's blood oxygen saturation level and pulse rate. The interface unit provides for communication between and control of the PCA unit and the pulse oximetry unit, and further provides an interface between the user and the system. When the pulse oximetry unit indicates to the interface unit that the patient's blood oxygen saturation level and pulse rate has reached a user-specified minimum, the interface unit initiates visual and audio alarms and controls the PCA unit by shutting off the PCA unit. The interface unit also contains communication ports, which the interface unit can use to send signals to external devices, such as to alert alerts medical personnel.

Document ES2584915 provides an automatic flow dosing device for oxygen therapy equipment. The invention describes an automatic flow dosing device for oxygen therapy equipment comprising: a means of estimating the extent of physical activity of patient that may be carried by the patient; a processing means in communication with the estimating means and with the oxygen therapy equipment, which is set up to receive from the estimating means said estimation of the physical activity of the patient, to determine the oxygen level required by the patient, and to transmit to the oxygen therapy equipment an order corresponding to the oxygen level required by the patient.

U.S. Pat. No. 6,314,957 provides a portable apparatus for domiciliary and ambulatory oxygen therapy intended for people suffering from respiratory insufficiencies and treated by administering gaseous oxygen to correct the gas contents in their blood, in particular the carbon dioxide and oxygen contents. The system includes an air compression device; a concentrator device allowing gaseous oxygen having a purity of 50 to 99% to be produced; an oxygen liquefaction device; an accumulation and storage device for the oxygen liquefied by the liquefaction device; a liquefied oxygen warming and vaporizing device; and a gas transport section for conveying the oxygen warmed and vaporized by the warming/vaporizing device to a gas delivery interface connected to the upper airways of a user.

Document CN203451219 (U) describes a utility model that discloses a portable oxygen generator. The portable oxygen generator comprises an oxygen generator shell with an inner cavity which is separated into two spaces by a middle separation plate, wherein an air filter, an air compressor, a control main board, a driving board and a battery are arranged on one side of the middle separation plate; a combined adsorption tower, a combined solenoid valve and an oxygen pulse valve are arranged on the other side of the middle separation plate; the air filter, the air compressor, the combined solenoid valve and the combined adsorption tower are connected by pipelines in sequence along an air inlet direction; an oxygen pulse valve is connected with an oxygen outlet pipeline of the combined adsorption tower; the combined adsorption tower comprises two adsorption towers and an oxygen tank arranged between the adsorption towers; the combined solenoid valve comprises a valve plate, and a solenoid valve, a safety valve and a silencer which are arranged on the valve plate. According to the portable type oxygen generator, the combined adsorption tower is driven by electric power and oxygen is prepared by a pressure swing adsorption circulating process to be used as a continuous oxygen source for individual oxygen therapy and oxygen health care; meanwhile, an integrated design of the combined adsorption tower and the combined solenoid valve is adopted so that the volume of the oxygen generator is reduced greatly and the portable type oxygen generator is convenient to carry.

U.S. Pat. No. 6,470,885 provides a method and apparatus for controlling oxygen delivery to a person is disclosed. In one embodiment, the method includes receiving a goal blood-oxygen saturation level, measuring an actual saturation level of the person, determining a breath rate of the person, sensing a period of inhalation by the person, and delivering oxygen during inhalation by moving a valve to an oxygen delivery position for a calculated period of time based upon the actual saturation level as compared to the goal level of the person's blood-oxygen content. One embodiment of an apparatus comprises an open-loop breathing system including a control valve for controlling the flow of oxygen from a source to the person, a pressure sensor for detecting a period of inhalation, an oximeter for measuring actual blood-oxygen saturation, and a processor for calculating the time the valve needs to be maintained in an open position to deliver oxygen.

Document US2002096174 presents the invention of a portable oxygen concentrator system adapted to be transported by a user. The portable oxygen concentrator system includes an energy source, an air separation device powered by the energy source and adapted to convert ambient air into concentrated oxygen gas for the user, at least one sensor adapted to sense one or more conditions indicative of the oxygen gas needs of the user, and a control unit interrelated with the air separation device and the at least one sensor to control the air separation device so as to supply an amount of oxygen gas equivalent to the oxygen gas needs of the user based at least in part upon the one or more conditions sensed by the at least one sensor.

Document PE20110495 refers to an equipment that captures medical data comprising sensors for capturing electrical signals of heart activity; a sensor for capturing the pulse oximetry signal for observing on screen the oxygen saturation in the blood; a keyboard for selecting the options for observing the cardiac or pulse oximetry signal; and a control device for data processing and control of the peripherals. The communication of the equipment with the medical center is in real time and is performed through the cellular telephone network, the equipment is portable, and it is powered by batteries or a power supply.

Document ES2469805 provides an oximeter sensor comprising: a light emitter for directing light to a patient; a light detector mounted to receive light coming from said patient; and a memory that storages coefficients for use in at least one formula for determining oxygen saturation, said coefficients including at least a first set of coefficients and a second set of coefficients, wherein the first and second sets of coefficients apply to different ranges of oxygen saturation values.

Document ES2024927 describes a medical biological data acquisition and transmission equipment which comprises data acquisition means, data control means, and transmission means, and is characterized because the data acquisition means comprise at least un first sensor for pulse and blood pressure, a second sensor for temperature and a third sensor for blood oxygen saturation; a keyboard to select the sensors; and a selection circuit which selects the sensor by pressing the corresponding key; the control means comprising a microprocessor by means of which normal operation and programmed automatic operation are possible. The sensors are arranged in an enclosure where the patent inserts a finger. A variation allows communication by any means of transmission without needing a network connection. Another variant includes a micro telephone and a magnetic card reader which allows remote data transmission.

However, the equipment and methods mentioned above are focused on the use and/or visualization of isolate variables, establishing independent controls which are exclusively or regulation or manual control.

Thus, what is needed is a modular device, tested and functional and must consider the medical specifications, specified ranges, and basic operating modes. It also must provide automatic regulation of provision of the amount of oxygen/air gas mixture to be administered to patients; this achieving a reduction of the incidence of damages and optimizing time of oxygen assistance to patients by reducing it to the minimum necessary. Furthermore, unnecessary alarms must be avoided, either due to patient's movement, own response of each patient, movement or loosening of the sensors, temporary saturation changes which exceed the limits and that return to normal, inadequate ranges that cause excessive alarms or, on the contrary, are not triggered, excessive or null manipulation of the nurses, and cancellation of alarms for different reasons, such as excessive false alarms.

### SUMMARY OF THE INVENTION

Due to the important care that should be given to oxygen saturation to sustain life, the present invention provides a device and system for measurement and reliable automatic control of oxygen dosage, which satisfies the requirements of each patient and also aida in the improvement through the control of the evolution according to the pathology of the patient.

The instant invention uses the interrelation of the variables that can be measured in the human body (oxygen saturation, heart rate, blood pressure, respiratory rate, temperature, etc.) to corroborate through software processing, the measurement of oxygen saturation and to maintain an adequate saturation level, in addition to the use of artificial intelligence, specifically an expert system that supports patient's recovery.

The present invention provides an intelligent automatic oxygen therapy equipment using as direct variable the monitoring of oxygen saturation (SpO2) by pulse oximetry, which is verified through the values and waveforms of the other signs acquired by the equipment. The device automatically controls the oxygen saturation value to keep it within the desired range through software processing that analyze all the variables that the equipment measures, and also considers the historical response of the patient to calculate how to supply the gas flow in the adequate amount and speed. The response of the patient is controlled in real time to modify on-line the control strategy according to the response obtained and extrapolating values to generate predictive alarms that allow acting efficiently. The main object is to provide reliability of the oxygen saturation measured value when using the information of the other measured variables.

The equipment of the invention avoids complications occurring due to lack or excess of oxygen saturation in the organism and the problems appearing when having false measurements.

The device/system of the invention performs an analysis of the measured variables and waveforms, applies software processing the measurements to detect events such as sudden changes that may occur due to patient's movements or for sensors out of place. It analyzes also the plethysmographic waveform which is compared against known waveforms from different pathologies to inform medical staff.

The system of the invention detects differences out of the normal range of the variables, showing the possible problem. It performs an analysis of the variables to apply alarm handling algorithms for their optimization, considering, among other things, high sensibility of the systems, improper calibrations, patient's movements, patient's response, and waveform out of range, inconsistence of values of the same variable coming from different sensors, loose sensors, or cancelled alarms.

According to an aspect of the invention, the intelligent automatic oxygen therapy system includes a device that allows dosing automatically the percentage of the oxygen/air gas mixture and the flow to be delivered to each patient through non-invasive oxygen therapy procedures.

According to another aspect of the invention, the system is based in monitoring the SpO2 through a pulse oximeter and in the automatic gas flow regulation to maintain both, the flow and the oxygen saturation within the ranges established by the medical staff.

According to yet another aspect of the invention, SpO2 values are validated by comparing other signs such as, but not limited to a) pulse oximeter heart rate with the corresponding ECG, b) the plethysmographic wave matching with ECG waves and their adequate forms compared with established standards; c) the changes of heart rate and respiratory rate according to the temperature of the patient.

According to still another aspect of the invention, the waveforms of the respiratory rate are compared with the capnographic waveforms, and the heart rate obtained by the NIBP. The equipment can also use the respiratory rate to collect all the related information and to act in the most reliable and synchronized way in the treatment with oxygen therapy.

According to one aspect of the invention, thanks to the implementation of an expert system, the comparison and analysis of all data allows the equipment to have artificial intelligence with the following functions, for example: automatic control of the administered amount of oxygen/air gas mixture, higher reliability in the control of parameters of automatic oxygen therapy, generation of more realistic and optimized alarms, directions on misplaced sensors, estimation of the evolution of each patient, optimization of oxygen/air gases supply, support to recovery or evolution control of the patient and minimum intervention.

According to another aspect of the invention, the equipment through artificial intelligence level 2 (expert system), allows to optimize the parameters of the equipment to auto-adjust to the requirements of each patient and to obtain the desired results in the oxygen therapy treatment.

According to yet another aspect of the invention, the equipment has a parameters configuration interface established by the medical staff. It has three operating modes: manual, automatic, and intelligent. This last mode includes the gradual decrease or increase of oxygen based on the historic responses of the patient, this cooperates with the evolution of the pathology and promotes respiratory independence.

### BRIEF DESCRIPTION OF THE DRAWINGS

Further features and advantages of the invention will become apparent from the following detailed description taken in conjunction with the accompanying figures showing illustrative embodiments of the invention, in which:

FIG. 1 shows an example block diagram showing the interconnection of the devices, according to the present invention.

FIG. 2 shows an example flow diagram of the solution needed to be implemented in a microprocessor-based system for an intelligent automatic oxygen therapy system, according to the present invention.

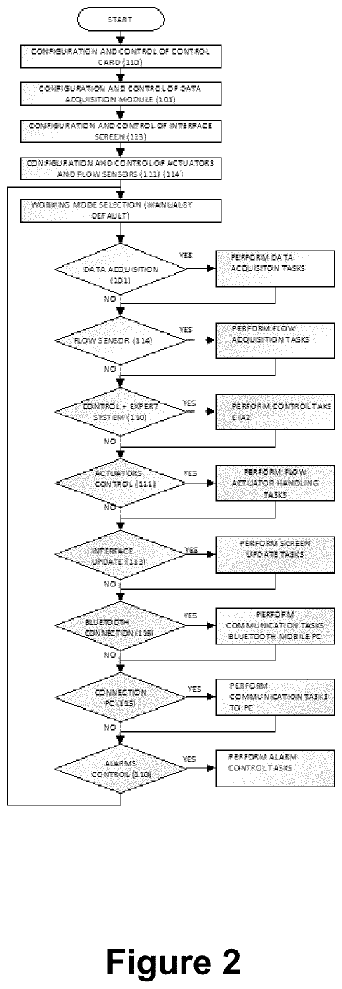

FIG. 3 shows a diagram of possible cases of behavior of a recovering patient indicating limits and control used in the analysis for the behavior of alarms, according to the present invention.

Throughout the figures, the same reference numbers, and characters, unless otherwise stated, are used to denote like elements, components, portions or features of the illustrated embodiments. The subject invention will be described in detail in conjunction with the accompanying figures, in view of the illustrative embodiments.

Other objectives, advantages, and new characteristics of the invention will become more evident with the detailed description that follows, when taken together with the drawings that are attached. The description illustrated in the drawings is an example and is not limited to the shown figures.

### DETAILED DESCRIPTION OF THE INVENTION

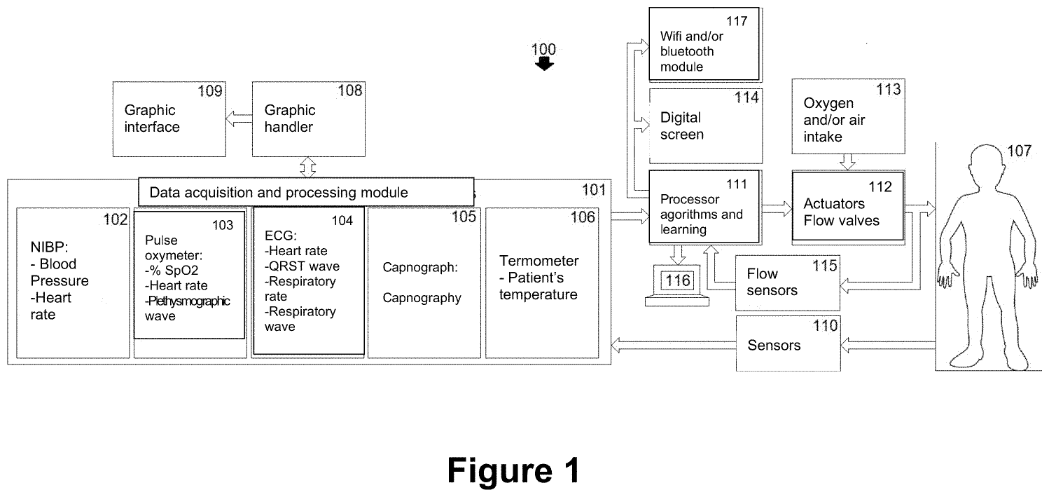

The system (100) shown in FIG. 1 is illustrative. In some embodiments, one or more components may be optional. In some embodiments, the system may include additional components not shown in the drawing. For example, in some embodiments, the system may take other signs available in the system to include them in the calculation algorithms, may have different ways of communication or may include different forms of artificial intelligence. Besides, in some embodiments, the parts may be placed or organized in a form different than those shown in the example drawing appearing in FIG. 1.

It is performed a description of embodiments of examples of methods, systems, and apparatus that allow a device to control automatic or intelligently the oxygen therapy, by managing the oxygen flow or is mixture with air (112), based on the primary monitoring of the oxygen saturation 402 obtained through an optical sensor. This measurement is confirmed with other signs to improve reliability and effectiveness of oxygen therapy. For that purpose, the equipment has a configuration interface (114) of the parameters in some embodiments includes: oxygen saturation 402 limits, maximum control-minimum control-maximum alarm-minimum alarm-waiting time-performance time-return bands to normal values. These values are established by the medical staff according to the situation of each patient (107). Besides, in some embodiments includes the configuration of basic information of the patient (age: premature, child, adult, senior, personal data), allows to fix acceptable ranges of the parameters may be performed from the equipment (114) parameters and (109) graphics. In some embodiments it is monitored by computer (116) directly connected or using a wired or wireless communication network (117), besides, in some embodiments through an application for smartphone or portable devices. In addition, in some embodiments, it is monitored by real-time video for remote monitoring of the treating physician.

In some embodiments, three operation modes are available: manual, automatic and intelligent. Manual mode allows visualizing the measurements and the gas flow being administered to the patient; the opening of the supply valve (112) is manually performed without intervention of the control. According to the information of the SpO2 value, the system activates visual/audible alarms when leaving the safe range established in the parameters; however, in this mode no corrective action is taken. In general, oxygen manual dosage delivers to the patient an amount higher than needed, and the body takes longer to get rid of this support.

In some embodiments, in automatic mode the equipment performs the oxygen therapy control based on the calculations made by the processor (111), controlling the gas flow through solenoid valves (112) automatically at the right time and speed to compensate the need of the patient until he reaches the adequate saturation margin according to the scheduled performance band (hysteresis) (114). In turn, in some embodiments, if saturation exceeds the maximum established limit, the gas dosage decreases until saturation enters the stabilizing band (hysteresis). The alarm limits (maximum and minimum) are configured so that if there is a high decompensation that has not been quickly stabilized within the control bands, the visual/audible alarms are activated (114) to alert the medical staff. In some embodiments, the professionals based on the information provided by the system (109) and (114) may evaluate the situation and take a drastic measure to stabilize the patient, if necessary, optimizing the system alarms (111).

Regarding the control decision making (111) the received SpO2 value is analyzed (103) and data and graphics of other measured variables are confirmed (101), by verifying that the obtained values of the optical sensor (110), are commensurate with the patient's condition (107). This way, the equipment in some embodiments dismisses measures which are lost or altered by movements, light, or positioning which affect the pulse oximetry equipment, among others, according to the algorithms programmed in the equipment (111).

Besides, in some embodiments this mode analyzes the information of the sensors which is processed in several acquisition and data processing modules (101). All the waves are compared using patterns to determine the reliability of measurements; for example, the plethysmographic waveform is compared against a wave pattern through the correlation algorithms (111) and it is determined the reliability of the oxygen saturation measurement.

Within the automatic mode there are five control options, which are: Oxygen Only, Constant Flow/Variable FiO2, Variable Flow/Constant FiO2, Variable Flow/Variable FiO2, Weaning Mode.

Within these options the equipment shall operate with the parameters established according to the medical criteria to provide the most appropriate treatment according to the patient's condition, to reach the improvement of the patient and to avoid secondary effects for excessive use of oxygen.

In the intelligent mode, in some embodiments, the equipment applies self-learning algorithms and artificial intelligence (111), which analyzes all variables of the system and the historical response of the patient, allowing lowering the oxygen levels to the strictly necessary level to help to “train” the lungs to stop depending on the oxygen or its mixture. Aside from this intrinsic functionality of the equipment, in some embodiments the device allows to select mode “weaning” for newborns. In this mode the control (111) includes a gradual and programmed reduction of oxygen or of the mixture with air, so that when the medical staff deems prudent (patient out of critical condition) the equipment shall provide a lower flow to stabilize the patient with a lower amount of oxygen or its mixture until oxygen therapy is withdrawn. This complements the lungs “training”, accelerating in a controlled way the recovery of the patients in less time, and minimizing the adverse effects of excess of oxygen (for example, ROP).

Similarly, in some embodiments if the equipment through the artificial intelligence algorithms detects that the self-recovery time of the patient is increasing, it alerts the doctor to take necessary actions by readjusting the corresponding parameters. If it is not enough, in some embodiments, it allows providing guides to the doctors to analyze certain pathologies that may be causing said condition.

Besides, in some embodiments, in the intelligent mode the equipment has a storage data system (1119 which generates a historic record of the behavior of the patient, considering the time lapses and the values that were out of the established acceptable range. This historic record has two uses; first one, in some embodiments allows the doctor to manage a record of the evolution of the patient of the medical history. In second place, it allows a feed back to the control system regarding the response times of the patients and understand their behavior through artificial intelligence algorithms (111), in some embodiments mode “weaning” may be applied to some adult patients to untie them from oxygen therapy assistance.

In the intelligent mode, in some embodiments, the information of sensors (101) processed in different acquisition and data processing modules (101) is analyzed. In some embodiments, as an example, the plethysmographic waveforms are analyzed and compared to vasoconstriction and vasodilatation wave patterns through validation algorithms (111), in some embodiments, for example, we proceed in a similar way with the values obtained from capnography to generate the best control strategy.

In some embodiments, the variables comparison applies algorithms described in their essence and that include the variations applicable to the same (111). In some embodiments, the system (100) has for example, independent modules of ECG (10), oximetry (103), non-invasive blood pressure (102), capnography (105) and temperature (106) which in some embodiments are the acquisition and data processing modules (101). This module delivers the appropriately scaled and filtered signs to the graphics handler module (108) and to the processor of algorithms and learning processor (111).

In some embodiments, in the intelligent mode, the alarms activation is subject to the verification of algorithms and artificial intelligence (111), in order to avoid false detections; this implies that in the cases when the variable is out of the established ranges in some embodiments it is considered first that the oxygen therapy system detects that the alarm is real and no due to events outside of the normal operation when confirming all variables, waveforms of the system, established limits and hysteresis bands. Besides, as a second step, it considers the history of the patient to analyze whether his answer to similar events represents or not an alarm and whether he can overcome autonomously the variation occurring, so that after verifying the above it determines whether an alarm will be triggered.

In some embodiments, in this mode there are also five control options: Oxygen Only, Constant Flow/Variable FiO2, Variable Flow/Constant FiO2, Variable Flow/Variable FiO2, and Weaning Mode.

Within these alternatives the equipment works intelligently based on the analysis of the data acquired from the behavior of each patient, limits fixed by the doctors, and recovery objectives.

In some embodiments, for any of these three operation modes: Manual, Automatic, and Intelligent, the system performs a validation of numerical values and waves (101).

In some embodiments, the waves are analyzed with a pattern wave through the calculation of the correlation between them (measurement which allows to express until when these two waveforms are similar).

After filtering the waves using traditional digital techniques, they are processed for validation (111). First, with the frequency value of each wave, at least three periods of them, are taken following the criteria of Nyquist theorem, they are averaged and normalized. Second, the standard wave is scaled to the period of the input waveform to initiate the analysis. Then the waves correlation method Discrete Wavelet Transform is used to determine whether or not the input waveform is reliable and that the measurements are not incorrect due to movements of patient, sensor displacement, or wrong positioning.

In some embodiments if the waves are within the correlation limit to be validated, the dynamic time warping algorithm (DTW) is used.

In some embodiments, if the waves are reliable then the pathologies analysis is performed. For example, in the Plethysmographic Waveform it can be determined through comparison with vasoconstriction or vasodilatation wave patterns, by displaying the tendency of the patient to these patterns. Besides, it is shown the average waveform of waves validated for the analysis. Similarly, from PQRST wave of the ECG it can be determined the ST-segment displacement to determine peaks and valleys and to deliver this information for medical analysis.

Considering that the variable controlled in the system is the percentage of oxygen saturation SpO2, its validation and reliability are very important, the system (100) in some embodiments, to confirm the validity of the measurement, performs, for example, the following considerations and validations: a) verifies that the plethysmographic wave has peaks and valleys with reliable values, b) compares that the values of heart rate obtained by ECG and the one obtained by the optical sensor, are within acceptable tolerances, c) that the time between the peaks of the plethysmographic wave is similar to the time of peaks of the ECG wave, d) verifies that sudden or out of SpO2 range variations are similar in values and waves of other signs, d) verifies that the graphic of respiratory sign is within the normal patterns, e) verifies the variation of the capnographic sign, f) analysis of the variation of the SpO2 which is out of normal ranges while the other measures are adequate, g) verifies that the values of the gas flow are within the programmed parameters; h) a sudden variation of any of the measures and/or waves. In some embodiments, after all these verifications, the system validates the value of measured SpO2.

All these conditions, in some embodiments, are evaluated by control algorithms (111) to provide intelligence and reliability to the SpO2 measured value and to act appropriately in the flow control (112) of the gas/air and/or oxygen coming from a source external to the system (113). This allows the system, in some embodiments, to control for example: gas flow, wait time for system stabilization, autoregulation by changing the variables of acting time and gas flow speed, auto-adjust of performance bands (hysteresis), generation of optimized alarms for the system and the patient, evolution of the patient, improper placement or loosening of sensors, evolution prediction, behavior extrapolation, and reference diagnosis.

In some embodiments, the medical staff initially fixes the maximum and minimum values of flow and gas concentration (108); limits of oxygen saturation; and, according to the patient's condition the oxygen therapy method is established, within the five methods of the equipment: 1. Oxygen Only; 2. Constant Flow/Variable FiO; 3. Variable Flow/Constant FiO2; 4. Variable Flow/Variable FiO2; and 5. Weaning Mode. Besides, the doctor fixes the estimated self-recovery times that may be initially expected, before the equipment starts working.

In some embodiments the system registers the self-recovery times of the patient in either compensation or decompensation depending on the patient's response during a period of analysis, these values are analyzed and averaged to establish the new self-recovery time. The increases or decreases of the self-recovery times are shown on screen through trend graphs of each one for the corresponding medical analysis.

Besides, the historical data of auto self-recovery times, flow, concentrations, and evolution of oxygen saturation (SpO2) influence the implemented expert system through Fuzzy logic; thus, continuously optimizing the flow rate and concentration of the gas mixture within the oxygen therapy method, either so that the patient's oxygen saturation remains within the established parameters and/or to reduce the gas dependence.

In some embodiments, the alarms are intelligently controlled, by determining the auto self-recovery times through the expert system and the comparison between the biomedical waves of the patient with the pattern waves, performing a quantitative and qualitative analysis of data to establish the optimal times of alarms activation and to increase reliability of the system.

In some embodiments, the intelligent alarms (111), depending on the behavior of the patient, act according to the following algorithm.

The system continuously verifies the reliability of the numeric values of vital sings of the patient among sensors, such as respiration per minute of ECG and SpO2 waves. In addition, each measured waveform is analyzed with standard waves, using the analysis algorithms described above to determine their correlation and thus their reliability.

If there is a difference in numerical values greater than a set percentage and the correlation of the waveforms in all variables is low, we wait for a time according to the self-recovery time and reanalyze the data. If the conditions are maintained, a sensor check alarm (movement, incorrect placement, or displacement) is presented.

If the correlation in the waveform of a single variable is low, we wait for a time shorter than the self-recovery time and then the wave is reanalyzed. If the correlation remains low, an alarm is generated from that sensor.

Once the data is validated, the system is verifying that the oxygen saturation is within the programmed limits, acting as follows in case of deviations from them:

1) If the patient recovers within the self-recovery time, no alarm is generated.
2) If the patient does not return to the hysteresis band within the self-recovery time, and the numerical values are in range and the waveforms have an adequate correlation in the automatic and intelligent modes, the system calculates the gas mixture supply values by means of Fuzzy logic.

The calculated values of the new set point are delivered to a PID controller that regulates the opening or closing of the air and oxygen valves.

Once the controller acts, two events can occur:

(a) If the patient tends to enter the programmed hysteresis values of oxygen saturation no alarm is generated.
b) If the patient moves away from the programmed values of the hysteresis band, an alarm is generated.

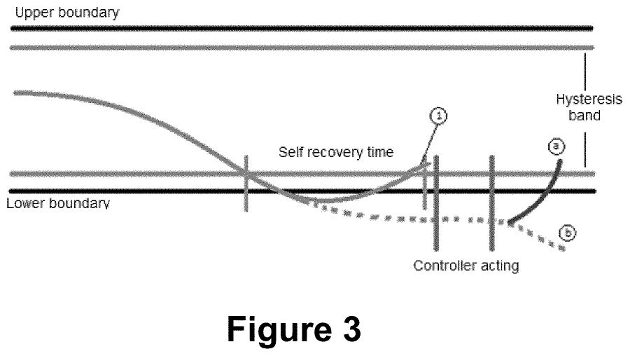

FIG. 3 exemplifies the above in the case of a decrease of the SpO2 measurement below the lower limit.

In addition to this, there are the system's own alarms such as low battery, power failure or lack of gas input, among others.

In some embodiments, for example, these control strategies and algorithms allow to provide the team with the necessary expertise, giving information support to the doctor for his diagnosis and oxygen therapy strategy for each patient by recalibrating the values of the performance bands.

In some embodiments, the system may be modular being the necessary base both, pulse oximetry and oxygen or its mixture flow control.

Although the present invention has been described herein with reference to the foregoing exemplary embodiment, this embodiment does not serve to limit the scope of the present invention. Accordingly, those skilled in the art to which the present invention pertains will appreciate that various modifications are possible, without departing from the technical spirit of the present invention.

I claim:

1. An intelligent automatic oxygen therapy system for a patient comprising:
at least one flow actuator configured to regulate a flow of air, oxygen, or a combination thereof provided to a patient;
at least one flow sensor configured to measure the flow of air, oxygen, or the combination thereof provided to said patient;
an oxygen saturation sensor configured to measure oxygen saturation levels of said patient; and
a processing unit configured to:
receive measurements from said at least one flow sensor;
receive oxygen saturation measurements from oxygen saturation sensor;
analyze the received oxygen saturation measurements against at least one of an oxygen saturation predefined range or measurements of at least one vital sign of the patient;
control said at least one flow actuator to adjust the flow of air, oxygen, or the combination thereof based on said analysis to maintain the oxygen saturation level of said patient within a desired range.

2. The intelligent automatic oxygen therapy system according to claim 1, wherein said at least one vital sign of the patient comprises an oxygen saturation level, a blood pressure, a volume of an organ or body of the patient, a heart rhythm, a concentration or partial pressure of carbon dioxide in respiratory gases, or a temperature.

3. The intelligent automatic oxygen therapy system according to claim 2, wherein said oxygen saturation sensor is a pulse oximetry (SpO2) sensor, said blood pressure is measured with a non-invasive blood pressure sensor, said volume of the organ or body of the patient is measured with a plethysmographic sensor, said heart rhythm is measured with an electrocardiogram (ECG) sensor, said concentration or partial pressure of carbon dioxide in respiratory gases is measured with a capnographic sensor, and said temperature is measured with a temperature sensor.

4. The intelligent automatic oxygen therapy system according to claim 1, wherein said at least one flow actuator is also controlled without said analysis.

5. The intelligent automatic oxygen therapy system according to claim 1, wherein said analysis is performed to determine at least one of sudden changes of values caused by movements of the patient, misplaced sensors, sensor failures, connection failures, or measuring errors.

6. The intelligent automatic oxygen therapy system according to claim 1, wherein said analysis is performed by an artificial intelligence level 2.

7. The intelligent automatic oxygen therapy system according to claim 5, wherein alert signals are generated when a problem with a sensor or a connection or a measuring error is detected.

8. The intelligent automatic oxygen therapy system according to claim 1, wherein said analysis comprises learning patterns based on at least one of a history of the patient, a pathology of the patient or interventions of medical staff so that said at least one flow actuator is actuated to optimize an oxygen automatic dosage.

9. The intelligent automatic oxygen therapy system according to claim 1, wherein said at least one flow actuator is coupled to a gas inlet or an oxygen inlet.

10. The intelligent automatic oxygen therapy system according to claim 1, wherein the measurements of said at least one flow sensor are displayed.

11. The intelligent automatic oxygen therapy system according to claim 1, wherein said processing unit is further configured to keep a record of at least one of a total oxygen consumption of the patient, sudden changes in measurements, or alterations in an oxygen monitoring pattern.

12. The intelligent automatic oxygen therapy system according to claim 7, wherein alert signals parameters are set by a physician.

13. The intelligent automatic oxygen therapy system according to claim 6, wherein at least one of possible patient pathologies or controlled flow ranges are suggested to a physician based on the analysis performed by said artificial intelligence level 2.

14. The intelligent automatic oxygen therapy system according to claim 1, wherein at least one of alarms, measurements received by the processing unit or analysis data are communicated to a remote device, displayed on a local device or a combination thereof.

15. The intelligent automatic oxygen therapy system according to claim 1, wherein said processing unit operates in:
a manual mode where said at least one flow actuator is manually actuated without said performing analysis;
an automatic mode where said at least one flow actuator is automatically actuated based on said analysis until the oxygen saturation level of said patient is within the desired range; and
an intelligent mode where said at least one flow actuator is actuated to train the lungs of the patient to stop depending on the oxygen or mixture thereof based on the analysis being performed with at least one of a self-learning procedure or an artificial intelligence procedure that analyzes all information received and generated by the processing unit.

16. The intelligent automatic oxygen therapy system according to claim 15, wherein the generation of alarms is controlled in said intelligent mode to reduce false measurements.

17. The intelligent automatic oxygen therapy system according to claim 16, wherein said alarms are generated only when measurements of the patient's vital signs are outside of predetermined ranges and the measurements have been validated by the intelligent mode.

18. The intelligent automatic oxygen therapy system according to claim 16, wherein the generation of said alarms is further controlled based on a patient's reaction time according to a learning scheme of a patient's behaviour to an oxygen therapy.

19. The intelligent automatic oxygen therapy system according to claim 1, further comprising
a display configured to display at least one of oxygen saturation levels or at least one vital sign of the patient;
a user interface configured to allow a user to configure the system; and
a communication module and an interface configured to communicate with at least one of a local device or a remote device.

20. The intelligent automatic oxygen therapy system according to claim 1, wherein said processing unit is further configured to determine a total volume of the air, oxygen, or the combination thereof delivered to the patient.
